(19) United States
(12) Patent Application Publication
(10) Pub. No.: US 2010/0022415 A1
(43) Pub. Date: Jan. 28, 2010

(54) ACTIVE CARRIER, ITS PRODUCTION AND ITS USE

(75) Inventors: Laszlo Puskas, Szeged (HU); Eszter Molnar, Pecs (HU)

Correspondence Address: Olson & Cepuritis, LTD., 20 NORTH WACKER DRIVE, 36TH FLOOR, CHICAGO, IL, 60606, US

(73) Assignee: AVICOR KET., Szeged (HU)

(21) Appl. No.: 12/310,306

(22) PCT Filed: Aug. 21, 2007
(86) PCT No.: PCT/HU2007/000077

§ 371 Date: Sep. 16, 2009

(30) Foreign Application Priority Data
Aug. 22, 2006 (HU) P0600668

(51) Int. Cl. C40B 50/00; C07F 7/02

(52) U.S. Cl. 506/23; 549/215; 544/229; 556/410

(57) ABSTRACT

Active carrier that consists of a carrier base (1) and attached linkers containing activated linker functional groups. The active carrier contains two or more different activated linker functional groups. A process for the production of the active carrier that comprises a carrier base (1) and linkers containing activated linker functional groups. Steps of the process: a) binding of a linker containing one or more linker functional groups to the carrier base (1), through the carrier functional groups; and b) reacting the linker functional groups of the linkers bound to the carrier base in step a) with two or more different activating reagents simultaneously. Use of the active carrier, during which the surface of the active carrier is contacted with one or more solution of small molecules.

The present invention pertains to an active carrier for surface immobilization of low molecular weight compounds, the method for the production of such active carrier, and the use of such active carrier.

A new protein, whether it is human, animal, plant, viral or other natural or artificial, brings many new challenges for researchers. Proteins can be characterized in numerous ways, e.g. by their sequence, molecular weight, spatial structure and no less importantly by the natural or artificial ligands they bind, including the parameters of binding.

As a result of the mapping of the human genome, several thousands new human proteins are waiting to be identified and characterized. The identification of proteins as potential drug targets is one of the most important challenges of pharmaceutical research in the years to come. Numerous methods have been created for the identification of the relationship between certain disease states and proteins encoded by the human genome. Such methods include, for example, comparative 2D gel electrophoresis (A. Görg, Proteomics, 2000, Jul. 3), as well as isotope labeling methods combined with mass spectrometry (S. P. Gygi and co., Proteomics, 2000, Jul. 31).

In numerous cases, the above methods for the identification of the relationship between the given protein and the disease state yield no result, for example, because their application is difficult or they can not be used at all in cases where protein concentrations are low, and the simultaneous separation and detection of a large number of proteins can also be realized only with difficulty. Furthermore, it is also known, that establishing a connection between a protein and a known disease state is only one of the first steps of pharmaceutical research. It is followed by the identification of such small molecules that are able to modify the function of the protein advantageously from the human aspect, and then follows, through many steps, the development of any of these small molecules into pharmaceutical drug.

At the same time, the binding of a small molecule (i.e. low molecular weight molecule) to a given protein validates its future treatment as a drug target-molecule through the fact that it is able to bind to the protein at all. The preliminary selection of the molecules that are able to bind to a given drug target-molecule limits the number of molecules that may act as inhibitors or inducers, i.e. those that may become potential pharmaceutical drugs. Furthermore, expression analyses can establish the influence of the binding on the disease state, and, by these analyses, the function of the protein can also be verified. (F. Darvas and co., Curr. Med. Chem. 2004 December; 11 (23):31 19-3145).

The development of combinatorial chemistry and parallel syntheses, where molecular building blocks are attached to a central structural element in the large number of their possible combinations, facilitated the production of a large number of new small molecules and the analogues of already known effective pharmaceutical drug molecules, whose number is in the order of magnitude of tens to hundreds of thousands. As a result of this, the creation of molecule libraries with several hundreds of thousands to million members became possible.

For the screening of molecule libraries with such high number of members, different screening systems are available, which, however, have to be adapted, with consideration of the characteristics of the given protein, to the cell system that expresses or to the other biological system that contains the given protein. The development of such a system often takes several months, even a whole year. In addition, the screening of such a large library requires a modern robotic system, which makes these methods costly. Several drug target and protein families are known whose screening has not been solved yet or can not be adapted to high-throughput systems. Because of these, the classification and general characterization of new and already known proteins—the application of screening systems that are realized by the analysis of interactions based on affinity with a large number of small molecules—is now at the forefront of research.

The use of synthetically produced organic small molecules—generally performed in the above mentioned screening systems—is usually included as an integral part in the identification and functional validation of proteins encoded by newly discovered genes. This new approach is called ‘chemical genomics’ or ‘chemical proteomics’ in the literature.

The affinity methods that are based interactions between proteins and small molecules—which are also suitable for the isolation and identification of proteins—have already been applied in the form of small molecules bound to cellulose or various polymers. The single proteins in the protein-mixture flown through affinity columns containing this kind of loads leave the column separated as a consequence of their different flow velocity that depends on their strength of interaction with the small molecules bound to the column. This way, the binding and non-binding proteins can be separated.

There have been several attempts to make the method suitable for mass and parallel application, yet these methods have shown only a slight development. The technique called affinity chromatography in a comprehensive name is time-consuming, its demand for small molecules is significant, and its parallel application can be realized only with difficulty.

A revolutionary development was reached with the high density binding of small molecules to micro-sized plates *(slides). The small molecules are bound to the plate (slide) in a well-defined arrangement in the form of a plane matrix. In each point of the plane matrix, there is an immobilized cluster consisting of a given single type of molecule. These micro-sized plane matrix carriers, as well as the arrangement of small molecules in plane matrix form is also called ‘microarray’ as it is well known for those skilled in the art. An advantageous attribute of microarray in contrast with classic high-throughput screening is that, since the compounds bound to the carrier are located side-by-side, a high number of measurements can be performed under completely identical conditions, thus the comparison of the outcoming results enables one to draw more accurate conclusions. A further advantage of microarray is that it facilitates the especially useful application of high-capacity, robotized technology. The quantity of molecules bound to the microarray is small, because the surface area is small, therefore micromolar quantities of small molecules are sufficient for the preparation of the given points of as much as several hundreds of microarrays. A further advantage is, that not only the production requires small quantities of the molecule, but, during screening, the quantity of test molecules (in a given case, target proteins) is also small (1-5 μg), which is a significant economic advantage in comparison with the affinity-interaction based screening systems applied hitherto.

The technology of binding onto cellulose or glass was first developed for the production of DNA-microarrays, during which different DNA-molecules are bound to the same carrier. This method is widely applied in hybridization analyses now. Several chemical methods have been developed for the binding of DNA and other oligonucleotides to carriers (L. Hackler Jr. and co. Mol. Divers. 2003; 7 (1): 25-36.). The above technology made a significant contribution to the faster than expected determination of the human genome. Since then, the development of protein microarrays for the determination of protein-protein interactions and expression levels has also been initiated. (MacBeath G, Schreiber SL. Science. 2000 Sep. 8; 289 (5485): 1760-1763.).

Beier M. and Hoheisel J. D. (Nucleic Acid Res. 27(9) 1970-1977 (1999)) describe a method for the derivatization of solid carriers for the creation of covalent bonds on DNA-microarray, during which, the surface of the carrier is increased with a branching-structured linker molecule. The synthesis of this linker molecule, or simply linker, requires a four-step reaction. Before immobilization, the surface that contains the linker, and, by this, it is functionalized, is activated with a further activating reagent, such as PDITC (phenylene diisothyocyanate), DSC (Disuccinimydil carbonate) or DMS (dimethylsuberimidate). As a result of the activation, the free end of the linker becomes suitable for covalently binding DNA. Although different DNA molecules can be effectively attached to the surface formed this way, the stabilities of the bonds differ greatly, and from these differences, the conclusion could be drawn that covalent bonds are formed only at some places, between an inner amino group of a DNA-molecule and the linker.

Attempts for binding small molecules to carriers have been made only recently. These solutions are based on different chemical mechanisms and binding strategies. The bindings of sample molecules can be of various strengths, so their stabilities can also be different. Most generally, glass-made microscope slides are used for the binding of chemical molecule libraries, for the production of chemical microarrays by analogy to DNA-microarrays. One of these types of binding methods has been realized by the researchers of a German company, Graffinity, who bound organic molecules onto gold surface through thiol groups (see: German patent No. DE 100 27 397).

Researchers have developed several kinds of chemical reagents, and linkers carrying them, that are able to bind small molecules to carriers (see: for example, Japanese patent No. JP 3032740 or PCT publication No. WO-01/01143, or U.S. Pat. No. 6,824,987). Currently, mainly chemically modified microscope plates are used for the production of chemical microarrays. For the facilitation of small molecule binding to carriers, small molecules generally have to be provided with a suitable functional group that helps their binding to the functional groups (e.g. to the previously mentioned thiol group) formed on the carrier. This way, the molecules can be bound through a linker molecule containing a thiol, amino or carboxyl group. More accurately, the addition of a thiol, amino or carboxyl group to the molecule to be bound creates the possibility for the small molecule to bind to a carrier containing, for example, a thiol, maleimid, amino, carboxy, ester, epoxy, bromocyane or aldehyde functional group. The binding between the small molecule provided with the functional group and the free end of the linker bound to the carrier is realized through the formation of thio, ether, ester, amide or amine bonds. **So, for example, in the USA patent description, U.S. Pat. No. 5,919,523, the production method of solid carriers coated with glycane or with polymers that can be used in solid-phase synthesis of peptides, oligonucleotides or low molecular weight organic molecules or ligand arrays is disclosed, where the polymer contains amine, carboxyl and/or hydroxyl functional groups.

One of the disadvantages of most methods that use mercaptosilane- or epoxysilane-coated, silanized solid carriers (see. for example, U.S. Pat. No. 5,919,626) is that the molecules bound by this method are located close to the surface of the solid carrier, thus they are not easily accessible for the probes to be tested for an interaction, and, as a result, the number of specific bindings may decrease.

The Hungarian patent No. P021091, discloses the synthesis of a new carrier or carrier-set that is applicable for the binding of pharmaceutical drug and drug-candidate small molecules through such reactive groups, that are located on branching-structured connecting linkers. The new carriers according to the invention are able to covalently bind molecules having various functional groups of various lengths. The solid carriers produced according to this method can be used for the preparation of various microarrays and for the application of these carriers in molecular agro-chemistry, biology, biotechnology, and pharmacology.

By using the split-mix method of combinatorial chemistry (Á. Furka and co. Int. J. Pept. Protein Res, 1991, 37, 478), a quite large number of compounds can be produced in a mixture, and then these can be effectively bound to glass surface with a technique called ‘microprinting’ by using thiol-reactive anchoring groups (maleimid) (G. MacBeath and co., J. Am. Chem. Soc., 1999, 121, 7967). Although the method is suitable for the analysis of interaction between a protein and a large number of molecules, the binding of the compounds takes place randomly, so the identification of molecules in a given position is complicated.

In addition, the method has to be mentioned, where the synthesis of combinatorial molecule libraries is performed on the surface of a polypropylene layer* in a way that the location of the molecules on the surface is known (D. Scham and co., J. Comb. Chem., 2000, 2, 361), and then, it is followed by the application of this combinatorial library for biological screening.

The formation of combinatorial chemical microarrays, i.e. the application of libraries created by combinatorial chemical methods onto microarrays, is based on the application of such solid carriers that are able to bind various small molecules. Bound compounds can be applied in numerous, advantageous ways, for example: combinatorial chemical microarrays can be especially useful tools in different molecular biological and pharmaceutical developmental research, since, this way, the chemical surrounding of a given lead molecule can be easily mapped.

The area of application of chemical microarrays may extend to the analysis of interactions between known pharmaceutical molecules or other small molecules and newly discovered proteins, and so, for example, to the classification of new proteins. In this case, binding proteins are separated from nonbinding proteins, and then they are removed from the plate or the affinity column, and the binding proteins are identified by a suitable method (e.g. MS-MS; see: M. J. Dutt and K. H. Lee. Proteomic analysis, Current Opinion in Biotechnology, 2000, 11, 176-179).

The application of chemical microarrays facilitates high-throughput biological screening in such a way, that upon binding the protein the effective compounds show fluorescent emission in the given position, and since the topology of the microarray is available, the active compound(s) can be immediately identified.

During the production of affinity columns used in affinity chromatography, small molecules are immobilized on glass or polymer beads or other suitable column loads. The loads created in this way are very similar in their structure to the chemical microarrays described in detail above, but they are different in that, in the case of affinity columns, the bound compounds are not located in a plane matrix form, but are fixed on the surface of the loads. From the protein solution flown through the column containing the load with immobilized small molecules on its surface, the proteins that are able to bind to the small molecules immobilized on the load, bind to them, and then, by state-of-the-art methods the binding proteins can be eluted from the load. The number of molecules immobilized on the load is several magnitudes higher than that of the molecules bound on chemical microarrays, therefore these techniques of affinity chromatography can also be used for the effective separation of larger quantities of proteins.

Affinity chromatography also facilitates applications similar to chemical microarray. In this case, a certain compound is bound to the surface of each load element, e.g. bead, furthermore, each load contains a reporter molecule that characterizes the given load element. The mixture of loads prepared in this way is mixed with a solution of target-molecules, e.g. a protein solution. The loads that bind the target molecule can be separated from the loads that do not bind the target molecules with procedures known by those skilled in the art (e.g. on the basis of spectral characteristics or mass). After this, by the identification of the reporter molecule, the identification of the molecule that had been bound to the given load becomes possible. So, during the application of this method, the loads, each fitted with a single type of reporter molecule, are the analogues of the points of chemical microarray, as, in this case, the immobilized compound can be identified with the help of the reporter molecule of the load, and in the case of chemical microarray, the topology makes the identification possible.

The disadvantage of the above described active carriers is that the type of molecules that can be bound to a given carrier is determined by the functional group of the carrier, or, in case of application of a linker, by the functional group of the linker. A further disadvantage of the hitherto known carriers is that if the molecule is bound to the carrier through one of its groups that would interact with the target protein in solution phase, then the protein will not bind this immobilized molecule during the screening.

The goal of the present inventors was the creation of an active carrier to which a molecule library can be bound whose diversity is higher than that of the libraries of hitherto known carriers.

The present inventors realized that if, during the production of the active carrier, instead of applying one kind of activating reagent for the creation of the activated linker functional group, they perform the reaction with a mixture of two or more activating reagents, then, as a result of the even distribution shown by the activating reagents in the reaction mixture, all activating reagents will be equally present in each and every point of the carrier base during the reaction, thus, there will be activated linker functional groups created by every type of the activating reagents in every point. In this way, it becomes possible to form several kinds of linker functional groups evenly distributed on a chemical microarray, thus such an active carrier will be suitable for the binding of a molecule library with higher diversity than if there would be only one functional group on the surface. In the present case, this higher diversity molecule library does not only mean that several different kinds of molecules can be bound, but, in addition, it also means that the same molecule can be bound through its several different groups, thus the same molecule will have several different, free surfaces during screening.

The goal set by the present inventors has been solved by the creation of such an active carrier, that contains linkers having different kinds of activated linker functional groups attached to the carrier base, by this, several kinds of molecules can be bound to the same active carrier. In this way, such molecule library can be bound to an active carrier made according to the present invention that covers a chemical area larger than those used in cases of any known carriers.

In accordance with the above, the present invention discloses an active carrier, which consists of a carrier base and attached linkers containing activated linker functional groups, which active carrier is characterized by that it contains two or more different kinds of activated linker functional groups.

In one embodiment of the present invention, the active carrier contains two, three, four or five different activated linker functional groups.

In another embodiment, the active carrier contains two or three different activated linker functional groups.

In a further embodiment, the material of the carrier base is glass, natural polymer or artificial polymer.

In another embodiment, the material of the carrier base is glass.

In another embodiment, the material of the carrier base is polypropylene.

In another embodiment, the linker is straight chained or a branch chained, and contains primer and/or secondary and/or tertier amino group(s).

In another embodiment the linker is straight chained or branch chained polyamine.

In another embodiment, the linker contains maximum twenty substituted or unsubstituted linker functional groups.

In another embodiment, the linker is according to the general formula 1.

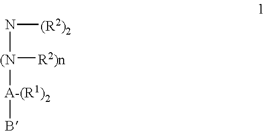

Wherein

the meaning of A is C or Si;
the meaning of B′ is C or O;
the meaning of R¹ is independently H, hydroxyl, —C₁-C₂₀-alkyl or —OC—C₀-C₂₀-alkyl, —C₃-C₆-cycloalkyl, where the alkyl advantageously is methyl, ethyl or propyl;
the meaning of R² is independently H, —C₁-C₂₀-alkyl, —OC—C₀-C₂₀-alkyl, —C₃-C₆-cycloalkil, nitrile, isocyanate, thiocyanate, isothiocyanate, which, in a given case, with the exception of H, can be substituted with one or more groups selected from the following, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, —(N(R³)CH₂)ₘ—N(R³)₂, halogenide, acid halogenide, where the halogenide can be chlorine, bromine, iodine;
the meaning of R³ is independently H, —C₁-C₂₀-alkyl, —OC—C₀-C₂₀-alkyl, —C₃-C₆-cycloalkil, nitrile, isocyanate, thiocyanate, isothiocyanate, which, in a given case, with the exception of H, can be substituted with one or more groups selected from the following list, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, —(N(R⁴)CH₂)ₘ—N(R⁴)₂, halogenide, acid halogenide, where the halogenide can be chlorine, bromine, iodine;
the meaning of R⁴ is independently H, —C₁-C₂₀-alkyl, —OC—C₀-C₂₀-alkyl, —C₃-C₆-cycloalkil, nitrile, isocyanate, thiocyanate, isothiocyanate, which, in a given case, with the exception of H, can be substituted with one or more groups selected from the following list, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, halogenide, acid halogenide, where the halogenide can be chlorine, bromine, iodine;
the value of n can be 0, 1, 2, 3, 4, 5, 6, 7, 8, 9 or 10;
the value of m can be independently 0, 1, 2, 3, 4, 5, 6, 7, 8; and the linker is attached to the carrier base through the atom marked B′, advantageously to the surface Si atom of glass carrier, or to the surface C or N atom of polymer carrier.

The invention also discloses the method for the production of the active carrier, which method comprises the following steps:

a) binding of a linker containing one or more linker functional groups to the carrier base through the carrier functional groups; and
b) reacting the linker functional groups of the linkers bound to the carrier base in step a) with two or more different activating reagents simultaneously.

In one embodiment of the present invention, the linkers are bound to the carrier surface by sililization*.

In one embodiment, the sililization is performed with 3,3-[2-(2-aminoethylamino)ethylamino]propyl-trimethoxysilane).

In another embodiment, the sililization is performed with N-(2-aminoethyl)-3-aminopropyl-trimethoxysilane.

In a further embodiment, the linker functional groups are reacted with two, three, four or five activating reagents simultaneously.

In a still further embodiment, the linker functional groups are reacted with two or three activating reagents simultaneously.

In one embodiment, activating reagents are selected from the following compounds, include, but not limited to: acrylic acid chloride, epichlorohydrin, chloroacetonitrile, chloroacetic acid chloride, bromoacetic acid chloride, chloroformic acid chloride, bromoformic acid chloride, chloroacetic acid anhydride, bromoacetic acid anhydride, iodoacetic acid chloride, iodoacetic acid anhydride, chloroformic acid anhydride, bromoformic acid anhydride, iodoformic acid anhydride, acrylic acid anhydride, 1,4-butanediol-diglycidil eter, chloroacetic acid isocyanate, bromoacetic acid isocyanate, iodoacetic acid isocyanate, acrylic acid nitrile, chloroacetaldehyde, bromoacetaldehyde, iodoacetaldehyde, 4-chlorobutyric acid chloride, 4-bromobutyric acid chloride, 4-iodobutyric acid, 4-chlorobutyric acid anhydride, 4-bromobutyric acid anhydride, 4-iodobutyric acid anhydride, chloroacetic acid isothiocyanate, bromoacetic acid isothiocyanate, iodoacetic acid isothiocyanate, 3-chloropropanic acid chloride, 3-bromopropanic acid chloride, 3-iodopropanic acid chloride, N-chlorocarbonyl isocyanate, phenyl diisothiocyanate, disuccinimdyl-carbonate, disuccinimidyl-oxalate, dimethyl suberimidate and 4-nitrophenyl chloroformate.

Further, the invention pertains to the application of the active carrier, during which the surface of the active carrier is treated with one or more solution of small molecules.

In one embodiment of the active carrier, different points of the surface of the active carrier are treated with two or more different solutions of small molecules for the production of chemical microarrays.

In another embodiment, chromatographic column loads are used as active carriers, and small molecules are bound to them for the production of affinity columns.

In the following part of the description the invention is expounded in detail with the help of drawings, which serves the purpose of elucidating the invention, however they, by no means, limit the scope of the invention.

The drawings:

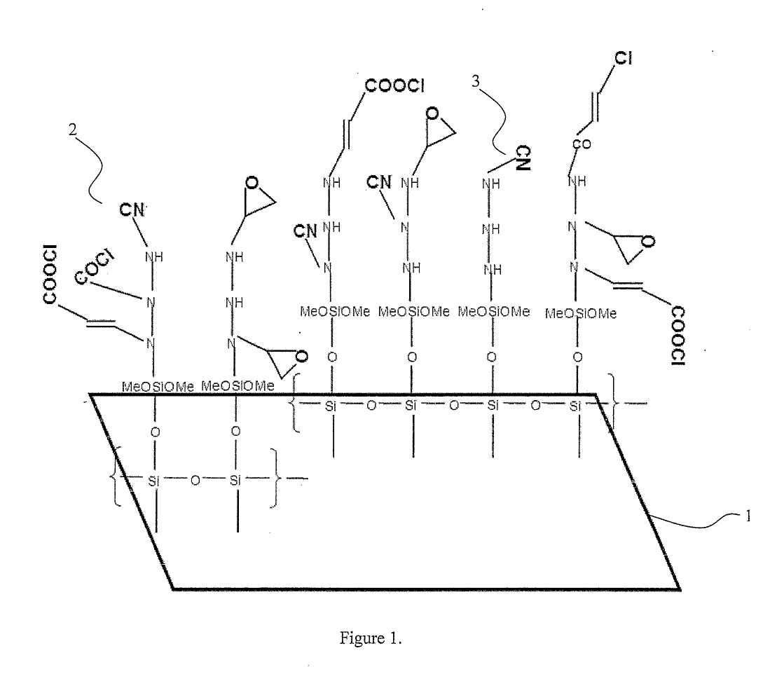

FIG. 1 shows a schematic picture of an active carrier

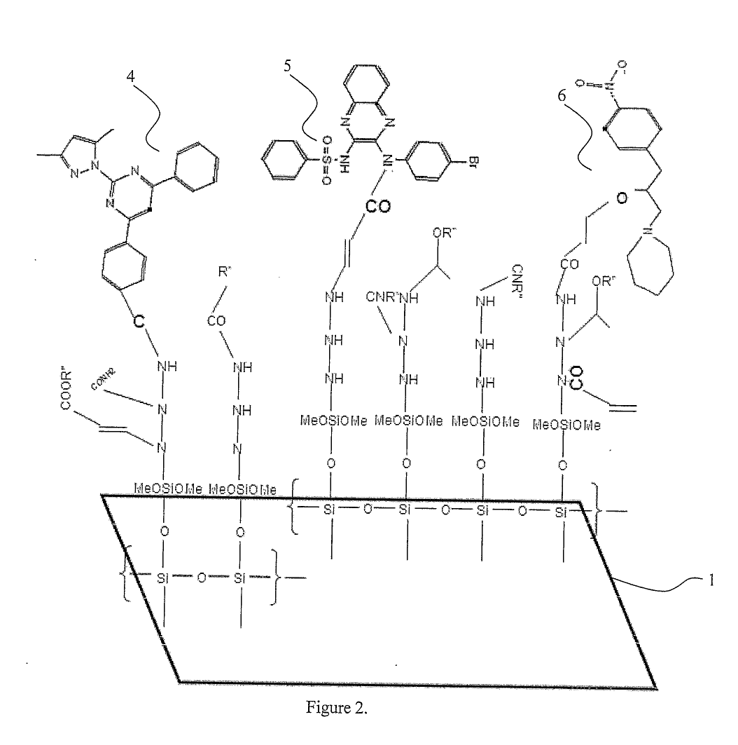

FIG. 2 shows a schematic picture of a chemical microarray

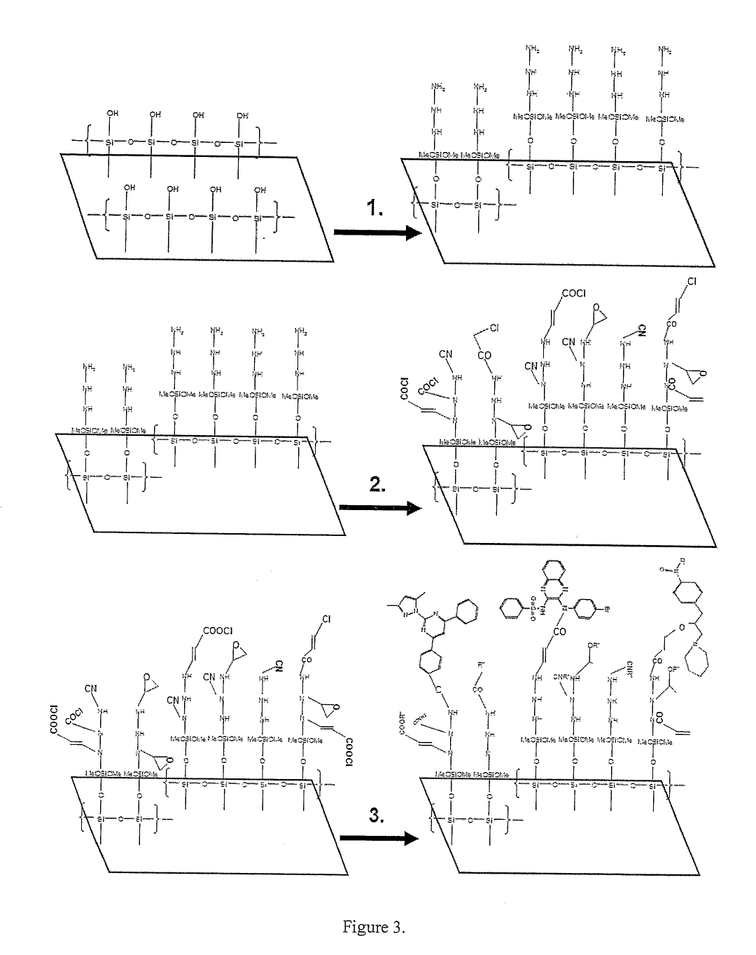

FIG. 3 shows the outline of the production scheme of chemical microarrays

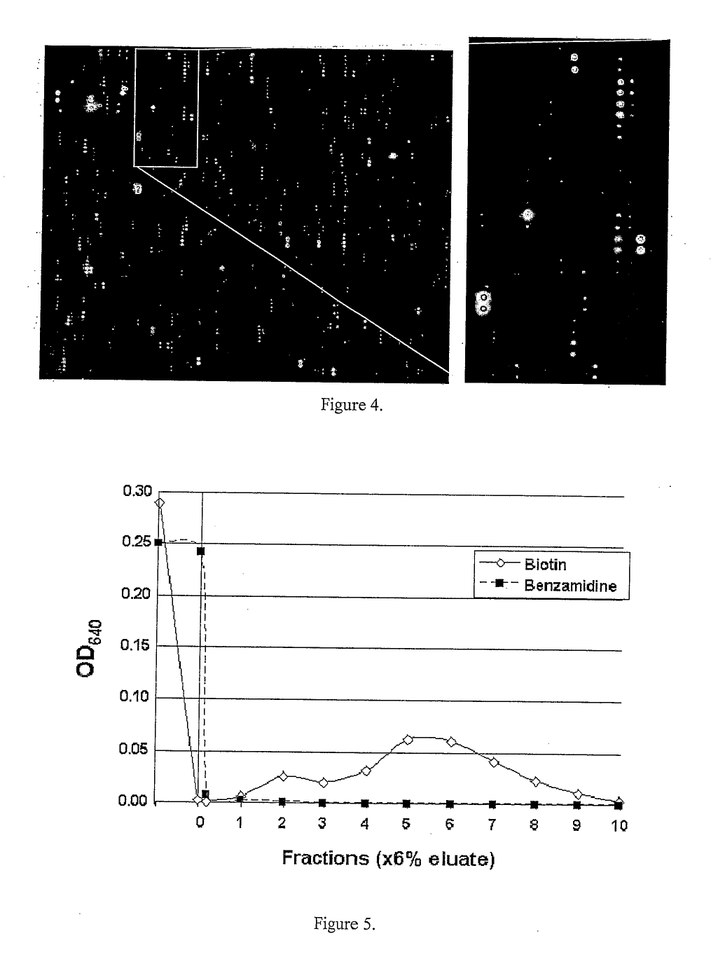

FIG. 4 shows the result of the interaction analysis of fluorescently labeled Proteinase K; and

FIG. 5 shows the results of affinity chromatographic analysis performed on biotinylated glass beads.

It will be understood that this invention is not limited to the particular methodology, protocols, reagents described. It is also to be understood that the terminology used herein is for the purpose of describing particular embodiments only and is not intended that this terminology should limit the scope of the present invention. The extent of the invention is limited only by the terms of the appended claims.

By the term ‘carrier base’, a macroscopic-sized solid body is meant, which contains carrier functional groups. In the light of the present description, carrier base can especially be glass-plate, glass bead, polymer plate, polymer bead. It is well known to the skilled artisans, that the geometrical parameters of the carrier base may vary according to the application, it may be plane, spherical or other form.

By the term ‘linker’, a chemical unit is meant, which connects two other chemical units through strong chemical bonds, for example: covalent bonds. Thus a linker ensures a certain molecular distance and, at the same time, a connection between the two linked chemical units. In the present description, by the term ‘linker’, a chemical unit is meant, which, on the one hand, binds to the carrier base, on the other hand, it binds to a sample molecule during the application of the active carrier according to the invention. Thus, as a result of this, the *sample molecule binds to the carrier base through the linker, and, in this way, it will be located in well-defined molecular distance from the surface of the carrier base. Within the frame of the present description, the term ‘linker’ also denotes the chemical unit, which, in accordance with the above, binds to the active carrier, but sample molecule does not yet bind to it, and this latter binding will be formed only in a later step of the formation of the active carrier.

By the term ‘functional group’, such chemical groups are meant, that are able to form strong chemical bonds, for example: covalent bonds, through reactions with other chemical units. In the context of the present description, functional groups may be situated on the carrier, the linker and, in a given case, on the sample molecules. In accordance with the present invention, the functional groups are given special emphasis, therefore, for the unequivocal definitions clear distinction is made between ‘the functional group of the carrier’, ‘the functional group of the linker’, and ‘the functional group of the activated linker’.

By the term ‘functional group of the carrier’, those functional groups are meant, which are part of the material used as the base material of the carrier, thus they are part of the carrier base. For example, in the case of glass carrier, the hydroxyl groups binding to the Si atoms are meant by the term ‘functional group of the carrier’. In accordance with the invention, the functional group of the carrier serves the purpose of binding the linker, i.e. of forming a strong chemical bond between the carrier base and the linker.

By the term ‘functional group of the linker’, those functional groups are meant, that are present on the linker prior to activation, i.e. those groups, that undergo some kind of a chemical reaction during the procedure of activation. In the case of application of linkers containing polyamine, the functional group of the linker, for example, include, but not limited to primer or secondary amine. In accordance with the invention, the functional group of the linker serves the purpose of facilitating the binding of various kinds of chemical units to the linker, and so the linker becomes suitable for the binding of small molecules.

By the term ‘functional group of the activated linker’, those functional groups are meant, that are formed as a result of the activation of the linkers, i.e. when chemical units of various kinds are bound to the functional group of the linker. These chemical units serve as the functional groups of the activated linker. Thus, the activated linker functional group is a functional group present on a substituted linker functional group. In the light of the present description activated linker functional groups are especially the following, include, but not limited to: thiol, amino, carboxyl, hydroxyl, phenyl, aldehyde, nitrile, keto, sulphonil, phosphate, ester, sulpho, pyridyl, pyrimidyl.

The term ‘microarray’ refers to micro-sized plane matrix carriers.

‘Chemical microarray’ is a kind of microarray, where on each point of the slide or plate an immobilized cluster composed of a given type of small molecule is located.

By the term ‘sample’ or ‘sample molecule’, that molecule is meant, which is bound to the active carrier by the linker through the activated linker functional group, i.e. these are the bound, or, in another word, immobilized molecules.

By the term ‘probe’ or ‘probe molecule’, the molecule is meant, whose binding to the sample is analyzed by observing whether the probe binds the sample bound to the active carrier or not. In case of the application of chemical microarrays, the probe is generally a macromolecule, for example, a protein.

By the term ‘immobilization’, the operation is meant, during which a molecule is bound to a solid carrier through a strong chemical bond, e.g. covalent bond. For those skilled in the art it is evident, that the terms immobilization, binding and anchoring refer to the same operation, and these terms are interchangeable. Within the frame of the present description, the inventors generally deal with the immobilization of small molecules on carriers, i.e. they immobilize the sample molecule on the carrier, in one aspect, through a linker.

That kind of carrier is called an ‘active carrier’, which contains activated linker functional groups bound to a carrier base through linkers.

A chemical microarray is generally composed of the following elements:

a) carrier base, which is microscopic-sized, and contains carrier functional groups;
b) in one aspect, linkers, which bind to the carrier base with the help of its carrier functional groups, and which, even after binding to the carrier base, have such free functional groups (i.e. with activated linker functional groups), that are required for the binding of sample molecules;
c) sample molecules that bind either directly to the carrier base with the help of its carrier functional groups, or to the linkers located on the carrier base, where clusters of a single type of molecules are located at each point of the carrier.

For skilled artisans it is apparent, that the linkers are not necessary but advantageous elements of a chemical microarray.

A chemical microarray functions the following way: The sample molecules immobilized on the carrier are contacted with the probe, for example, by dripping a suitable solution of a protein onto the surface of the chemical microarray. Then, this solution is washed off the surface using a buffer. On the points of the surface where such a sample is located, that can be bound by the protein in the immobilized state of the sample molecule, the protein remains bound there even after the wash off. Then, the protein bound in this way can be detected by methods well known in the art (e.g. in UV-light, by fluorescent technique etc.). One can determine which immobilized molecules can be bound by the observed protein on the basis of the topology of the chemical microarray (which contains the arrangement of sample molecules on the surface of the chemical microarray).

The present invention discloses an active carrier, which contains several kinds of activated linker functional groups, thus more kinds of small molecules can be immobilized on the carrier produced according to the invention, than on the carriers known hitherto.

FIG. 1 shows the schematic picture of the carrier according to the invention. The carrier base is constituted by a glass plate marked by reference mark 1. To the surface silicon oxide units of this carrier base, a substituted triamine is bound through a Si(OMe)₂ group. Of the amino groups of the triamine, one or more is substituted by different activated linker functional groups. Each amino group of the linker (provided with reference mark 2 on the figure) is single substituted by the following activated linker functional groups listed in order, closest to the carrier base first,* —CH₂═CH₂—COOCl, —COCl, és —CN. The figure highlites, that other linker units, on the same active carrier, have other activated functional groups. For example, the two secondary amino groups of the linker provided with reference mark 3 are not substituted, while its terminal amino group is single substituted by a —CN group.

On FIG. 2, one non-limiting embodiment of the present invention demonstrated. Herein, small molecules bind to the carrier through covalent bonds and through some activated linker functional groups of the carrier presented on FIG. 1. With reference number 4, a 2-(3,5 dimethyl-1H-pyrazol-1-il)-4,6-diphenyl pyrimidyn molecule binds to the active carrier through its —CN activated linker functional group, with reference number 5, a N-(3-(4-bromophenylamino)chinoxaline-2-il)benzylsulphonamide molecule binds to the active carrier through its —CH₂═CH₂—COOCl group, and with reference number 6, a 1-(4-nitrophenyl)-3-(piperidine-1-il)propane-2-ol molecule binds to the active carrier through its —CO—CH₂═CH₂Cl group. The groups used at blocking are marked R″, which are, in the present example, aliphatic alkylamines.

Blocking is necessary, because the proteins and the probe molecules would react aspecifically with the surface where small molecule samples are not bound. As a result of this, the background would increase largely, which would make reading and evaluation difficult or even impossible. Therefore the parts of the chemical microarray, where there are no sample molecules should be blocked (i.e. the activated linker functional groups are needed to be reacted), in such a way, that the reactive groups located there should loose their reactivity, i.e. should become inactive.

According to the state of the art, only such active carriers are known, that contains exclusively one kind of activated functional group. Because of this, only such molecules can be bound to these carriers, that contain a group which is able to react with the given activated linker functional group in a way, that strong chemical bond, for example, covalent bond is formed.

The present invention provides such an active carrier that simultaneously contains several activated linker functional groups on the surface of the carrier. This facilitates the binding of a molecule library more diverse than any known previously to an active carrier. Namely, because, in a given point of the active carrier, several kinds of activated linker functional groups are located, and, in this way, there is an increased chance for a compound from the solution of the given molecule applied to this point to bind to the carrier.

In addition, several activated linker functional groups can be located on a given linker unit of the active carrier according to the invention, and neighboring linkers may contain different functional groups (see FIG. 1). Namely, the recognition according to the invention comprises the fact, that the linkers are formed on a carrier base in such a way, that they carry numerous linker functional groups. In the example shown in FIG. 1, these linker functional groups are primer or secondary amino groups, however it is readily apparent for those skilled in the art, that other linker functional groups may also be applied. If the linker functional groups are activated with two or more activating agents simultaneously, then the single linker functional groups will be activated simultaneously but in different ways, and thus, different activated functional groups will be formed on the same active carrier.

On FIG. 3, the key steps of the process of the production of the active carrier according to the invention are presented by demonstrating the application of glass-plate as a carrier base. It is evident for those skilled, that not only glass, but other material, include, but not limited to natural or artificial polymers can be used as carrier base. In Step 1, linkers, that contain the linker functional groups, are bound to the surface of the carrier base. In the non-limiting example shown in the figure, triamino groups are attached to the hydroxyl groups on the surface Si atoms (for details, see Example 1) In this case, the carrier functional group is the hydroxyl group, and the linker functional group is the primer or secondary amino group. In Step 2, the linker functional groups formed in the previous step (in this case, the primer and secondary amines) are activated in such a way, that the surface prepared in Step 1 is reacted with different activating agents simultaneously. By this, different activated linker functional groups will bind to the linker functional group depending on the concentration ratios of the activating agents and on other reaction parameters, and the result will be a surface having several activated linker functional groups. Step 3 presented on FIG. 3 shows a schematic example for the application of the active carrier according to the invention. During this process, the surface formed in Step 2 is treated with the solutions of different molecules, and these different molecules will bind to different activated linker functional groups. Thus, by the method according to the invention, such an active carrier can be produced, that can bind even that kinds of molecules on the same surface, whose binding to the same active carrier was not possible according to the former state of the art because of their different chemical properties.

During the process according to the invention, generally microscope object-slide can be used most advantageously as a carrier base, whose sililization (i.e. the application of linkers according to Step 1 of FIG. 3) can be performed, for example, with 3,3-[2-(2 aminoethylamino)ethylamino]propyl-trimethoxysilane, or N-(2-aminoethyl)-3-aminopropyl-trimethoxysilane.

In the case of the application of primer or secondary amines as linker functional groups, the following activating agents are suitable for the activation of amino functions, and thus, for the formation of activated linker functional groups, include, but not limited to: acrylic acid chloride, epichlorohydrin, chloroacetonitrile, chloroacetic acid chloride, bromoacetic acid chloride, chloroformic acid chloride, bromoformic acid chloride, chloroacetic acid anhydride, bromoacetic acid anhydride, iodoacetic acid chloride, iodoacetic acid anhydride, chloroformic acid anhydride, bromoformic acid anhydride, iodoformic acid anhydride, acrylic acid anhydride, 1,4-butanediol-diglycidil eter, chloroacetic acid isocyanate, bromoacetic acid isocyanate, iodoacetic acid isocyanate, acrylic acid nitrile, chloroacetaldehyde, bromoacetaldehyde, iodoacetaldehyde, 4-chlorobutyric acid chloride, 4-bromobutyric acid chloride, 4-iodobutyric acid, 4-chlorobutyric acid anhydride, 4-bromobutyric acid anhydride, 4-iodobutyric acid anhydride, chloroacetic acid isothiocyanate, bromoacetic acid isothiocyanate, iodoacetic acid isothiocyanate, 3-chloropropanic acid chloride, 3-bromopropanic acid chloride, 3-iodopropanic acid chloride, N-chlorocarbonyl isocyanate, phenyl diisothiocyanate, disuccinimdyl-carbonate, disuccinimidyl-oxalate, dimethyl suberimidate and 4-nitrophenyl chloroformate.

The active carrier produced according to the present invention can be applied in several ways. One non-limiting example is that the active carrier can be used for the production of chemical microarrays by dripping solutions of small molecules on definite points of the surface of the active carrier made according to the above. This application can be performed manually or by using a robot. During application of the solutions, the topology of the microarray (the arrangement of which solution is dripped onto which point of the active carrier) is constructed either manually or automatically, according to instructions defined and programmed prior to application, by the computer that controls the application as well. Due to the present invention, chemical libraries with higher than ever diversity, i.e. covering higher chemical range, can be applied onto the same carrier, and thus chemical microarrays with larger than ever abundance can be produced. This way, molecules having completely different functional groups can be applied onto the same carrier.

The active carrier according to the invention can also be applied for the production of affinity column loads. For example, the active carrier according to the invention can be applied as a column load, for example, in the form of beads, when different small molecules are immobilized on each load. Although column loads containing different activated functional groups can be produced separately by hitherto known methods, the present method is more advantageous in comparison with previous methods, because it facilitates the production of such column loads within a single process, that contain several different activated linker functional groups, thus the uniformity of the loads is ensured, and, on the other hand, it eliminates the problem of reproducible mixing of different loads.

During the method according to the present invention, there is no need for the chemical modification of the probes to be bound, which makes the method much less costly in cases of the preparation of large quantity samples. During binding, there is no need for further chemical reactions, such as reduction. It is advantageous, because the samples containing groups sensitive to reduction can be bound to the solid carrier without any damage or modification.

In the following paragraphs, the invention will be elucidated by examples, which serve the purpose of better understanding, but they are, by no means, to be understood as limiting the scope of the invention.

### EXAMPLE 1

### Formation of Triamino-Silanized Surface on Glass-Plates

In this example, glass-plates are used as carrier base, on which one primer and two secondary amine linker functional groups are to be formed.

Commercially available microscope slides are left soaking in 10% aqueous NaOH (Molar Chemicals Ltd., Hungary, purity: >98.5%) solution, then it is washed with water, with 1% aqueous HCl solution (Molar Chemicals Ltd., Hungary), and then again with water until the washing solution reaches neutral pH. It is followed by drying the plates at room temperature. The etched glass plates prepared as above, are reacted with 3% N(2-aminoethyl)-3-aminopropyl-trimethoxysilane solution (ICN Biomedicals Inc. Aurora, Ohio), prepared in 95% aqueous methanol, for 2 hours. The plates are then washed with methanol, and then with water, dried and heat-treated at 105° C. for 15 minutes.

### EXAMPLE 2

### Formation of Branching-Structured Surface on Glass Plates

The surface formed in Example 1. and containing amino groups was incubated in 100 ml chloroform (Sigma-Aldrich) with 30 mmol acrylic acid chloride (Fluka) and with 30 mmol diisopropyl ethylamine for 2 hours. Then the plates were washed in 100 ml chloroform 5 times and dried at room temperature. The plates, then, were reacted with 1% tetraethylene pentamine (Sigma Aldrich) for 2 hours. The plates then were washed with methanol, followed by washing with water, dried and heat-treated at 105° C. for 15 minutes. The plates prepared by this method contained 15 free amino groups per linker (see the schematic drawing below), on which, by the method disclosed in example 3, such an active surface can be formed, that contains diverse activated linker functional groups.

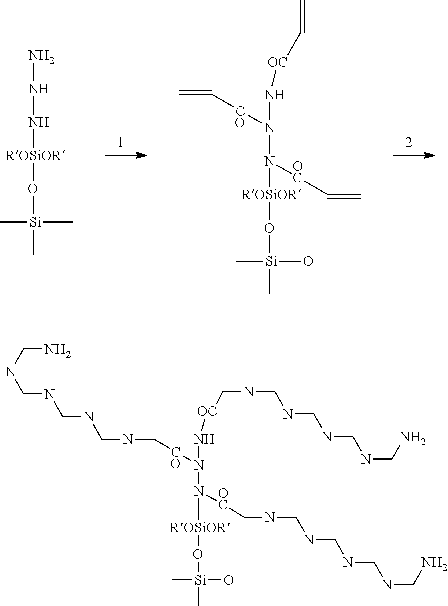

### EXAMPLE 3

### The Formation of Diverse Activated Linker Functional Groups on Triamino-Silanized Surface

Onto the surface prepared in Example 1 or 2, containing amino groups, a mixture of the following activating agents is applied: 8 mmol acrylic acid chloride (Fluka, Germany), 8 mmol epichlorohydrin, 8 mmol chloroacetonitryl, 8 mmol chloroacetic acid chloride (Fluka), 32 mmol diisopropyl ethylamine (ICN Biomedicals Inc., Aurora, Ohio) in 100 ml dichloroethane (Sigma Aldrich, Budapest, Hungary). This is incubated at room temperature for 2 hours. Then the plates are washed in 100 ml dichloroethane five times, and then dried at room temperature. As a result of this method, among other kinds, the following linkers can be formed:

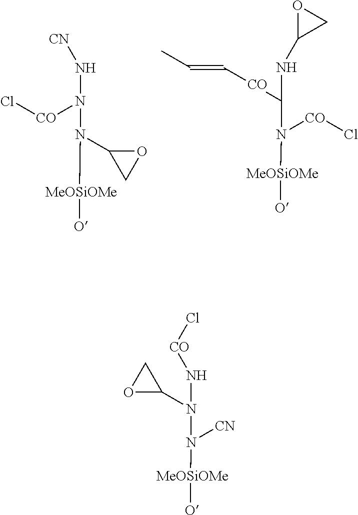

### EXAMPLE 4

### Production of High Density Chemical Microarray

Solutions of 10 mM concentration were prepared from various chemical compounds in dimethyl sulphoxide (DMSO) (Sigma Aldrich), then the solutions were transferred one by one into a 384-well microtiter plate (Greiner), knowing the position of each single compound. The microtiter plates were placed into a MicroGrid Total Array System (BioRobotics, England) robot. The chemically modified plates produced according to Example 3. were placed into the plate-holding unit of MicroGrid Total Array System (BioRobotics). The robot applied the solutions of small molecules from the microtiter plates onto the chemically modified plates (printed). The settings of the robot had been adjusted so that the distance between two printed spots would be 250 μm, and the diameter of the printed spots would be approx. 150-180 μm. The printing was performed at 50% humidity, at 16° C. temperature, and the plates were being cooled. After printing, the plates were incubated at room temperature for 2 hours. The incubation took place in a humid atmosphere to prevent drying of the applied drops. The quantity of the liquid applied to a single point by the robot was approximately 100 nl. After application of the sample molecules, the plates were washed with DMSO (3×100 ml), then with methanol (Molar Chemicals Ltd. Hungary, >99.7%). Blocking of the plates were performed in dimethylformamide containing 50 mM 6-amino-hexanol and 150 mM diisopropyletylamine at room temperature for 2 hours. The plates were then washed in dimethylformamide, methanol, and then with the aqueous solution of 1×SSC (0.1 M NaCl, 15 mM trisodium citrate), 0.2 w/w % SDS (sodium dodecyl sulphate), and then with water, and dried at room temperature. The complete plates were stored in dark at 4° C.

### EXAMPLE 5

### Compounds that Bind Only to Multiple Activated Surface

During their investigations, the present inventors noted, that there are small molecules that bind only to multiple activated surfaces, i.e. they can be immobilized only on a surface provided by the present invention. All compounds tested in this example were autofluorescing compounds.

Onto the surface produced according to Example 1, and containing amino groups, a mixture of activating agents chosen from the following compounds is applied: acrylic acid chloride (A), epichlorohidrine (B), chloroacetonitrile (C), chloracetic acid chloride (D). In Table 1, such compounds are presented as examples, that bound only to active carriers activated by more than one activating agents from the above. All molecules were tested on all possible surfaces. Only cases showing significant binding signals are shown in the table.

TABLE 1

| | Applied |
|---|---|
| Structural formula of molecule | activating agents |
| 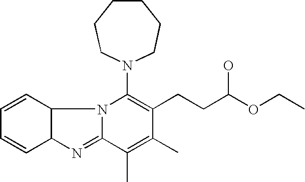 | ABCD |
| 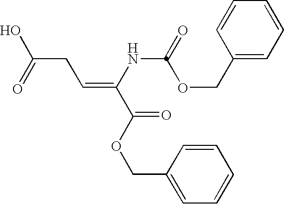 | BD, ABC, BCD, ABCD |
| 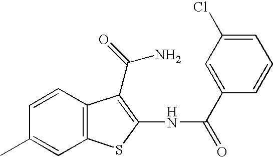 | BCD, ABC, ABCD |
| 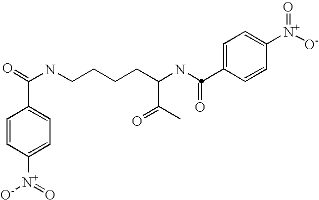 | BD, ABC, BCD, ABCD |
| 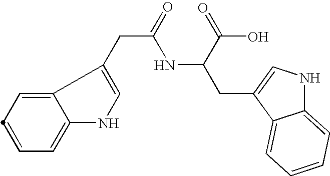 | ABC, BCD, ABCD |
| 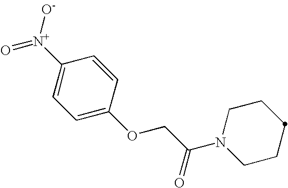 | ABCD |
| 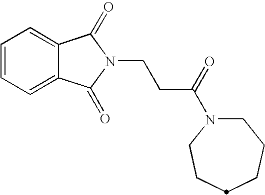 | ABCD |
| 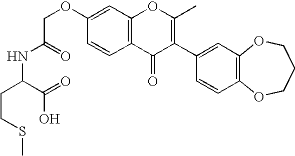 | ABC, BCD, ABCD |

It is clear from the above table, that, with the help of the carrier according to the present invention, such small molecules can be immobilized, that can not be bound, with appropriate effectiveness, to surfaces each containing only one activated linker functional group, but only the carrier according to present invention is suitable for their effective binding.

### EXAMPLE 6

### Examination of the Enzyme Proteinase K

Testing of the chemical microarray according to the invention was carried out with a serine protease protein, the enzyme Proteinase K. 1 mg Proteinase K (Sigma Aldrich, Budapest) solution was dissolved in buffered phosphate buffer [‘PBS’ ], then the protein was labeled with Cy5 fluorescent dye [Q15108, Amersham-Pharmacia] according to the protocol of SIGMA protein labeling kit (CSAA1, Panorama™ Ab Microarray Cell Signaling Kit). 5 μg of such fluorescent labeled protein was applied onto the surface of a chemical microarray that contained 8800 different chemical compounds each printed on the carrier in two distinct points, thus altogether 17.600 points were created by using a printing robot (BioRobotics, MicroGrid II, Cambridge, UK). FIG. 4 shows a portion of the microarray, and a selected section of it is also shown magnified. It is clearly seen, that the labeled serine protease interacted with several chemical compounds (brighter spots). During the course of further analysis, it was proven that certain compounds of those actually bind Proteinase K, in addition, some of them behaved as inhibitors, which was verified in a protease assay. Consequently, by using the chemical microarrays created by the present inventors, not only those compounds can be identified, that bind a given protein, but a significant portion of them behaved as inhibitors, thus the method is well utilizable in pharmaceutical research.

### EXAMPLE 7

### Preparation of Affinity Columns

In this example, controlled pore glass beads (Controlled Pore Glass) was used as the base of the carrier, on which one primer and two secondary amine linker functional groups were linked.

The commercially available Controlled Pore Glass (CPG-3Prime, USA) was left soaking in 10% aqueous solution of NaOH (Molar Chemicals Ltd. Hungary, purity: >98.5%), then it was washed with water, with 1% aqueous solution of HCl (Molar Chemicals Ltd., Hungary), then again with water on filter nutch* until the washing solution reached neutral pH. This was followed by drying the glass beads at room temperature.

The etched glass beads prepared as above were reacted with 3% N-(2-aminoethyl)-3-aminopropyl-trimethoxysilane (ICN Biomedicals Inc., Aurora, Ohio) solution prepared in 95% aqueous methanol for 2 hours. The glass beads, then, were washed with methanol, then with water, dried and heat-treated at 105° C. for 15 minutes.

By using the method disclosed in Example 3, active surfaces containing several activated linker functional groups were formed on the glass beads.

### EXAMPLE 8

### The Application of Affinity Column for the Detection of Biotin-Streptavidin Binding, and for Streptavidin Purification

Biotin was bound to glass beads prepared according to the method described in Example 7. in the following way: 10 mg biotin was dissolved in 1 ml DMSO (Sigma-Aldrich), then 200 mg of the activated glass beads were added to the solution and then it was stirred at room temperature for 2 hours. It was followed by washing the glass beads with 10 ml DMSO once, and with 100 ml methanol four times.

The biotinylated glass beads were added to 1 ml PBS, to which 50 μg of streptavidin labeled with Cy5 fluorescent dye was added (total dye: 0.3 OD₆₄₀/ml). The resulting solution was stirred, then OD₆₄₀ was measured again, the detected value was 0.003. This indicated that all labeled proteins (streptavidin) were bound to the biotinylated glass beads. Two chromatographic analyses were performed to verify the specific binding. During the first, 100 mg of the biotinylated glass beads was washed with biotin solutions with increasing concentrations, and in the second, the column containing biotinylated glass beads was washed with benzamidine solutions of increasing concentrations. The absorbance of the eluate was measured continuously (FIG. 5). The fluorescent labeled streptavidin could be eluted only with the biotin solution and not with benzamidine solution. This result confirmed the specificity of the binding.

The active carrier according to the present invention facilitates the binding of the same molecule, through different atoms or groups, to the same carrier. It is advantageous, because, in this way, a protein can approach the same molecule from several directions, thus, to the question, whether a certain molecule binds a certain protein or not, one can get a true answer with higher probability. In other words, the probability of “false negative” measurement results is decreased. This aspect can not be an issue for consideration with the active carriers constructed with former state-of-the-art techniques.

A further advantage of the present invention is that it facilitates the immobilization of molecules, whose binding to the carrier requires the simultaneous presence of several activated linker functional groups. This way, the invention provides solution for the immobilization of numerous small molecules whose binding to known active carriers according to the present state of the art has not been possible yet. In other words, the present invention facilitates the binding of not only the number of molecules that would bind to separate plates each with a single type of activated linker functional group, but significantly more molecules can be immobilized by this method, namely, those molecules, whose binding becomes possible only in case of the simultaneous presence of two or more activated linker functional groups.

Another advantage of the active carriers according to the present invention is that the microarrays produced from them do not necessarily contain only molecules similar in the chemical sense. The invention facilitates the binding of sets of molecules sorted according to different aspects to a given carrier, for example, according to a therapeutical area, molecular weight, ADME properties, requirements of Lipinski rule, other in silico, in vitro, or in vivo characteristics, or even grouping according to magisterial regulations (e.g. pharmaceutical registration, patent). Consequently, the active carrier according to the present invention is more advantageous than the presently known carriers not only because it is able to bind “merely” more kinds of molecules, but because this results in numerous new possibilities, among other things, for pharmaceutical research.

1. An active carrier for surface immobilization of low molecular weight compounds comprising a carrier base (1) and attached linkers containing activated linker functional groups, characterized by that the active carrier contains two or more different activated linker functional groups.

2. An active carrier according to claim 1, characterized by that said carrier contains two, three, four or five different activated linker functional groups.

3. An active carrier according to claim 2, characterized by that said carrier contains two or three different activated linker functional groups.

4. An active carrier according to claim 1, characterized by that the material of the said carrier base (1) is glass, natural polymer or artificial polymer.

5. An active carrier according to claim 4, characterized by that the material of the carrier base (1) is glass.

6. An active carrier according to claim 4, characterized by that the material of the carrier base (1) is polypropylene.

7. An active carrier according to claim 1, characterized by that said linker is straight chained or branch chained, and it contains primer and/or secondary and/or tertiary amino group(s).

8. An active carrier according to claim 7, characterized by that said linker is a straight chain or branch chain polyamine.

9. An active carrier according to claim 1, characterized by that said linker contains maximum twenty substituted or unsubstituted linker functional groups.

10. An active carrier according to claim 1, characterized by that said linker is according to the general formula 1 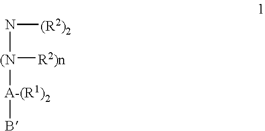
wherein
the meaning of A is Si;
the meaning of B′ is O;
the meaning of R¹ is independently H, hydroxyl, —C₁-C₂₀-alkyl or —OC—C₀-C₂₀-alkyl, —C₃-C₆-cycloalkyl, where the alkyl advantageously is methyl, ethyl or propyl;
the meaning of R² is independently H, —C₁-C₂₀-alkyl, —OC—C₀-C₂₀-alkyl, —C₃-C₆-cycloalkyl, nitrile, isocyanate, thiocyanate, isothiocyanate, which, in a given case, with the exception of H, can be substituted with one or more groups selected from the following, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, —(N(R³)CH₂)ₘ—N(R³)₂, halogenide, acid halogenide, where the halogenide can be chlorine, bromine, iodine;
the meaning of R³ is independently H, —C₁-C₂₀-alkyl, —OC—C₀-C₂₀-alkyl, —C₃-C₆-cycloalkyl, nitrile, isocyanate, thiocyanate, isothiocyanate, which, in a given case, with the exception of H, can be substituted with one or more groups selected from the following list, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, —(N(R⁴)CH₂)ₘ—N(R⁴)₂, halogenide, acid halogenide, where the halogenide can be chlorine, bromine, iodine;
the meaning of R⁴ is independently H, —C₁-C₂₀-alkyl, —OC—C₀-C₂₀-alkyl, —C₃-C₆-cycloalkyl, nitrile, isocyanate, thiocyanate, isothiocyanate, which, in a given case, with the exception of H, can be substituted with one or more groups selected from the following list, include, but not limited to: thiol, amino, carboxyl, hydroxyl, phenyl, aldehyde, keto, sulphonyl, phosphate, ester, sulpho, nitrile, epoxide, amidoalkyl halogenide, acrylamide, acid anhydride, isocyanate, isothiocyanate, azide, halogenide, acid halogenide, where the halogenide can be chlorine, bromine, iodine;
the value of n can be 0, 1, 2, 3, 4, 5, 6, 7, 8, 9 or 10;
the value of m can be independently 0, 1, 2, 3, 4, 5, 6, 7, 8; and the linker is attached to the carrier base through the atom marked B′, advantageously to the surface Si atom of glass carrier, or to the surface C or N atom of polymer carrier.

11. A process for the production of active carrier for surface immobilization of low molecular weight compounds comprising a carrier base (1) and attached linkers containing activated linker functional groups, characterized by that it consists of the following steps:
a) binding of a linker containing one or more linker functional groups to the carrier base through the carrier functional groups; and
b) reacting the linker functional groups of the linkers bound to the carrier base in step a) with two or more different activating reagents simultaneously.

12. A process according to claim 11, characterized by that said linkers are bound to the surface of the carrier by sililization.

13. A process according to claim 12, characterized by that the sililization is performed with 3,3-[2-(2-aminoethylamino)ethylamino]propyl-trimethoxysilane.

14. A process according to claim 12, characterized by that the said sililization is performed with N-(2-aminoethyl)-3-aminopropyl-trimethoxysilane.

15. A process according to claim 11, characterized by that said linker functional groups are reacted with two, three, four or five activating reagents simultaneously.

16. A process according to claim 15, characterized by that said linker functional groups are reacted with two or three activating reagents simultaneously.

17. A process according to claim 11, characterized by that the activating agents are selected from the following compounds: acrylic acid chloride, epichlorohydrin, chloroacetonitrile, chloroacetic acid chloride, bromoacetic acid chloride, chloroformic acid chloride, bromoformic acid chloride, chloroacetic acid anhydride, bromoacetic acid anhydride, iodoacetic acid chloride, iodoacetic acid anhydride, chloroformic acid anhydride, bromoformic acid anhydride, iodoformic acid anhydride, acrylic acid anhydride, 1,4-butanediol-diglycidyl ether, chloroacetic acid isocyanate, bromoacetic acid isocyanate, iodoacetic acid isocyanate, acrylic acid nitrile, chloroacetaldehyde, bromoacetaldehyde, iodoacetaldehyde, 4-chlorobutyric acid chloride, 4-bromobutyric acid chloride, 4-iodobutyric acid, 4-chlorobutyric acid anhydride, 4-bromobutyric acid anhydride, 4-iodobutyric acid anhydride, chloroacetic acid isothiocyanate, bromoacetic acid isothiocyanate, iodoacetic acid isothiocyanate, 3-chloropropanic acid chloride, 3-bromopropanic acid chloride, 3-iodopropanic acid chloride, N-chlorocarbonyl isocyanate, phenyl diisothiocyanate, disuccinimidyl-carbonate, disuccinimidyl-oxalate, dimethyl suberimidate and 4-nitrophenyl chloroformate.

18. The use of the active carrier for surface immobilization of low molecular weight compounds comprising a carrier base (1) and attached linkers containing two or more different activated linker functional groups, characterized by that the surface of the active carrier is contacted with one or more solutions of small molecules.

19. The use according to claim 18, characterized by that different points of the surface of the active carrier are contacted with two or more different solutions of small molecules for the production of a chemical microarray.

20. The use according to claim 18, characterized by that chromatographic column loads are used as active carriers and small molecules are bound to these for the production of affinity columns.
